(19) United States
(12) Patent Application Publication
(10) Pub. No.: US 2026/0017009 A1
(43) Pub. Date: Jan. 15, 2026

(54) Device for Assisting a User to Relieve Themselves

(71) Applicant: Andrew L. Pratt Living Trust dated October 26, 2016, Park City, UT (US)

(72) Inventor: Andrew Pratt, Park City, UT (US)

(73) Assignee: Andrew L. Pratt Living Trust dated October 26, 2016, Park City, UT (US)

(21) Appl. No.: 19/266,147

(22) Filed: Jul. 10, 2025

Related U.S. Application Data
(60) Provisional application No. 63/669,693, filed on Jul. 11, 2024.

(51) Int. Cl. G06F 3/16; A61M 21/00; A61M 21/02

(52) U.S. Cl. CPC G06F 3/165; A61M 21/02; G06F 3/162; A61M 2021/0027; A61M 2205/3561

(57) ABSTRACT

A device is discussed for assisting a user to relieve themselves, the device comprising: a memory in which at least one audio file is stored, the at least one audio file including a urination file comprising audio resembling a person urinating and/or a defecation file comprising audio resembling a person defecating; a device speaker, an actuator to be actuated by a user, and a controller that, upon actuation of the actuator, controls the device speaker to play the urination file and/or the defecation file.

### BACKGROUND

Sufferers of conditions such as Paruresis (shy bladder syndrome), Parcopresis (shy bowel syndrome), enlarged prostate, benign prostate hyperplasia, irritable bowel syndrome (IBS) or overactive bladder may have difficulty in relieving themselves comfortably by urination or defecation. Paruresis, at least, is named in the Americans with Disabilities Act Amendments Act of 2008 (ADAA) as a covered medical condition. Such conditions can be distressing and significantly impact the life of the sufferer. A solution is needed to help those with such conditions to be able to relieve themselves comfortably.

### SUMMARY

Embodiments of the invention include a device for assisting users to relieve themselves.

According to an aspect, there is provided a device for assisting a user to relieve themselves. The device may comprise:
a memory in which at least one audio file is stored, the at least one audio file including a urination file comprising audio resembling a person urinating and/or a defecation file comprising audio resembling a person defecating;
a device speaker;
an actuator to be actuated by a user, and
a controller that, upon actuation of the actuator, controls the device speaker to play the urination file and/or the defecation file.

The device may comprise one or more lights. The one or more lights may be of any suitable color or may be able to change color. The one or more lights may be illuminated when the actuator is actuated. This may give a visual signal to a user that the device is sufficiently charged. This may help a user with troubleshooting if they cannot hear an audio file when the actuator is actuated. In some examples, the light and/or an additional light, may be illuminated to indicate that a hearing device, such as a hearing aid, earphones or headphones is connected with the wireless technology device, such as through a Bluetooth® connection. In other examples, the light and/or an additional light, may be illuminated to indicate when the battery charge is low, or when the battery charge is sufficiently high to power the device.

The urination file may comprise an audio loop of a person urinating. The defecation file may comprise an audio loop of a person defecating. The audio loop may be created using Ambient Sound Engineering and/or Looping Audio Technology.

The actuator may be a first actuator. The at least one audio file may comprise the urination file and the defecation file. The device may comprise a second actuator to be actuated by the user.

The controller may, upon actuation of the first actuator, control the device speaker to play the urination file. The controller may, upon actuation of the second actuator, control the device speaker to play the defecation file.

Where there are two or more actuators on the device, when more than one actuators are actuated simultaneously, the controller may control the device speaker to play only one of the urination file and defecation file. The controller may determine whether the first actuator or the second actuator was actuated first, and play the corresponding audio file of the respective actuator which was actuated first. Once the actuator which was actuated first is released, the controller will control the device speaker to play the audio file corresponding to the actuator which was actuated second if the respective actuator is still actuated.

The first actuator may comprise a different shape and/or color to the second actuator. The different shape of the first actuator and the second actuator may refer to the three-dimensional shape. The three-dimensional shape of the actuator may include its externally visible two-dimensional shape as viewed by a user, and its surface texture including, for example, recesses and protrusions on the surface of the respective actuators. For example, the first actuator may have surface protrusions which spell “Pee” in Braille, and the second actuator may have surface protrusions which spell “Poo” in Braille. The different shape of the first actuator and the second actuator may refer to the size of the actuator.

The different colors of the first actuator and the second actuator may include, for example, the first actuator being in a shade of yellow, and the second actuator being in a shade of brown. The shades of the colors may be selected by taking into account color blindness around the world by, for example, selecting shades of each color that least affects those with color blindness.

The at least one audio file may be played on the device speaker while the actuator is actuated and stopped when the actuator is released. The at least one audio file is played on the device speaker when the actuator is actuated and stopped with the actuator is actuated a second time.

The at least one audio file may comprise a female file comprising a female urination file and/or a female defecation file. The female urination file may comprise a sound resembling a female urinating in a female toilet. The female defecation file may comprise the sound resembling a female defecating in a female toilet. The at least one audio file may comprise a male file comprising a male urination file and/or a male defecation file. The male urination file may comprise a sound resembling a male urinating in a male toilet. The male defecating file may comprise a sound resembling a male defecating in a male toilet. The male toilet may include a urinal or a toilet in a stall.

The sound resembling a female urinating in a female toilet may include the sound which may be heard in the vicinity, such as within the stall area of a public toilet. The sound resembling a male urinating in a male toilet may include the sound which may be heard in the vicinity, such as within a bathroom with urinals. The sound resembling a female defecating in a female toilet may include the sound which may be heard in the vicinity, such as within the stall area of a public toilet. The sound resembling a male defecating in a male toilet may include the sound which may be heard in the vicinity, such as within the stall area of a public toilet. This accounts for the variation in normal noises that a person might expect to hear in their usual encounters in public toilets.

The controller, upon receiving a signal from the actuator which is different from the actuation of the actuator, may switch between the female file and the male file, such that upon actuation of the actuator, either the female file or the male file is played on the device speaker.

In other words, the first actuator may be actuated in a predetermined rhythm, switching sequence or length of time to enable toggling between a male urination file and a female urination file which is preloaded in the memory. In other words, the second actuator may be actuated in a predetermined rhythm, switching sequence or length of time to enable toggling between a male defecation file and a female defecation file which is preloaded in the memory. A screen on the device may indicate which audio file has been selected for playing or whether male files or female files have been selected for playing. An audible cue may be played when toggling between male files and female files to indicate that a toggle signal has been received. This may be preprogrammed into the controller.

The controller may be connected to a portal with a wireless technology device disposed on the device, wherein the controller receives a signal from the portal to select alternative audio files to be stored on the memory.

The wireless technology device may include cellular technology or a short-range wireless technology device may comprise, for example, Bluetooth®, Wifi®, Near Field Communication (NFC) technologies. The device may link up with hearing aid suppliers by using shared APIs, to allow playback of all audio sounds directly into hearing aids, rather than through a Bluetooth connection.

The controller may be connected to a portal with a wireless technology device disposed on the device. The controller may receive a signal from the portal to select the volume of the device speaker.

The device may comprise a volume adjusting actuator which can be actuated to adjust the volume directly on the device. The first actuator and/or the second actuator may be actuated in a predetermined volume adjusting sequence such that the controller converts the first actuator and/or the second actuator into volume adjusting actuators such that subsequent actuation of the first actuator and/or second actuator results in the controller controlling the volume of the device to change. Further actuation of the first actuator and/or the second actuator in the predetermined volume adjusting sequence may convert the first actuator and/or the second actuator back to their original function.

The controller may be connected to a portal with a wireless technology device disposed on the device. The controller may receive a signal from the portal to select the female file or the male file to be stored on the memory.

According to another aspect, there is provided a device for assisting a user to relieve themselves. The device may comprise:
a memory in which at least one audio file is stored, the at least one audio file including a urination file comprising audio resembling a person urinating and/or a defecation file comprising audio resembling a person defecating;
a wireless technology device;
an actuator to be actuated by a user, and
a controller that, upon actuation of the actuator, controls the wireless technology device to transmit the at least one audio file to a remote speaker.

The urination file may comprise an audio loop of a person urinating. The defecation file may comprise an audio loop of a person defecating.

The actuator may be a first actuator. The at least one audio file may comprise the urination file and the defecation file. The device may comprise a second actuator to be actuated by the user.

The controller, upon actuation of the first actuator, may control the wireless technology device to transmit the urination file to the remote speaker. The controller, upon actuation of the second actuator, may control the wireless technology device to transmit the defecation file to the remote speaker.

The first actuator may comprise a different shape to the second actuator.

The at least one audio file may comprise a female file comprising a female urination file and/or a female defecation file. The female urination file may comprise a sound resembling a female urinating in a female toilet. The female defecation file may comprise the sound resembling a female defecating in a female toilet. The at least one audio file may comprise a male file comprising a male urination file and/or a male defecation file. The male urination file may comprise a sound resembling a male urinating in a male toilet. The male defecating file may comprise a sound resembling a male defecating in a male toilet. The male toilet may include a urinal or a toilet in a stall.

The device may comprise two through holes, each through hole for receiving a respective keyring. The two through holes may be on opposing sides or ends of the device. Having two through holes for receiving keyrings allows for one of the keyrings to be used for general keys, and the other keyring to be used to link a bathroom key. For people suffering from conditions which require urgent access to bathrooms, having a separate keyring for a bathroom key, which is easily accessible, can be important to reaching a bathroom in time.

The controller, upon receiving a signal from the actuator which is different from the actuation of the actuator, switches between the female file and the male file, such that upon actuation of the actuator, either the female file or the male file is played on the device speaker.

The device may comprise a rechargeable battery and a charging port for charging the battery.

According to another aspect, there is provided a device for assisting a user to relieve themselves. The device may comprise:
a memory in which at least two audio files are stored, the at least two audio files including a urination file comprising audio resembling a person urinating and a defecation file comprising audio resembling a person defecating;
a wireless technology device;
a device speaker;
a first actuator to be actuated by a user and a second actuator to be actuated by a user, wherein the first actuator comprises a different three-dimensional shape and/or color to the second actuator, and
a controller that:
   upon actuation of the first actuator, controls the device speaker to play the urination file or controls the wireless technology device to transmit the urination file to a remote speaker, and
   upon actuation of the second actuator, controls the device speaker to play the defecation file or controls the wireless technology device to transmit the urination file to the remote speaker.

The various embodiments described in the summary and this document are provided not to limit or define the disclosure or the scope of the claims.

### BRIEF DESCRIPTION OF THE FIGURES

FIG. 1 schematically shows an isometric view of a first example device.

FIG. 2 is a block diagram of a system within the first example device.

FIG. 3 schematically shows an isometric view of a second example device.

FIG. 4 is a block diagram of a system within the second example device.

### DETAILED DESCRIPTION

A device 100, 200 is disclosed for assisting a user to relieve themselves. The device 100 will provide near-immediate (if not immediate) medically non-invasive and risk-free relief to millions of sufferers. For example, the user may be a sufferer of Paruresis (shy bladder syndrome), Parcopresis (shy bowel syndrome) or any other condition which results in a difficulty in relieving themselves, particularly in public restrooms. These conditions typically present as a result of anxiety or discomfort with urinating or defecating in public restrooms, or in the vicinity of any other person, even in the home.

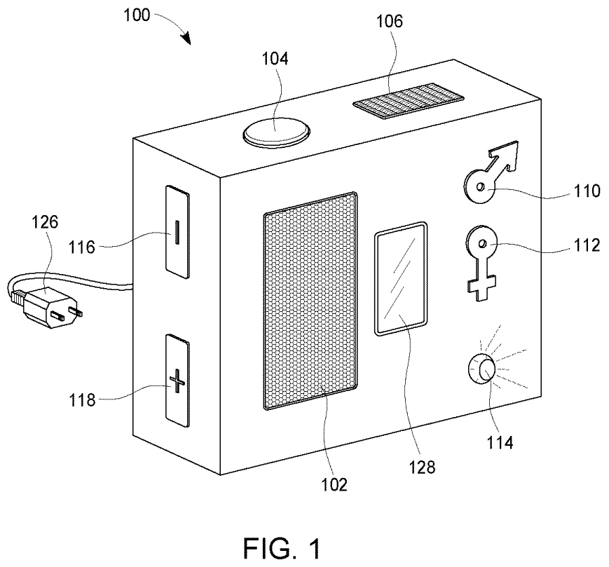

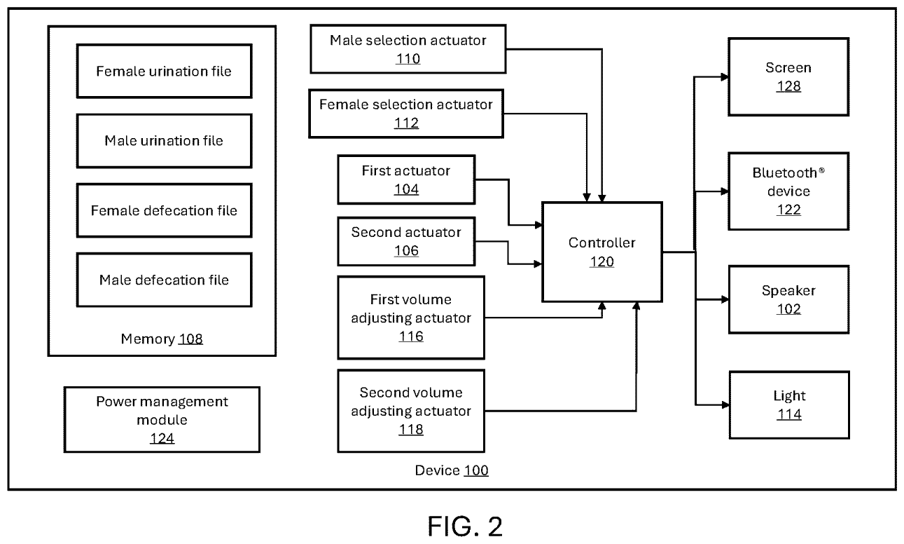

FIG. 1 schematically shows a first example device 100 and FIG. 2 shows a block diagram of the components of the first example device 100 which may be mounted on or a part of a printed circuit board (PCB). The PCB may be a single sided PCB, or a double sided PCB for space efficiency. In other examples, In-Mold Electronics (IME) or Flexible Printed Circuits (FPCs) may be used instead of a PCB. Broadly, the example devices 100, 200 are loaded with audio resembling the sound of urinating and/or defecating which a user can listen to on demand to assist them to comfortably relieve themselves.

The first example device 100 may be suitable for use in a bathroom within a home environment, with a permanent power supply, and may comprise a power cable for connecting to a power outlet in a home to power the device 100.

In this example, the device 100 comprises a device speaker 102, a first actuator 104, and a second actuator 106. The first actuator 104 and the second actuator 106 are distinctly separate. The first actuator 104 and the second actuator 106 are configured to be actuated by a user. In some examples, there may be only one actuator, or there may be more than two actuators. The first actuator 104 in this example is in the form of a first button which can be depressed to actuate it. The second actuator 106 in this example is in the form of a second button which can be depressed to actuate it. In some examples, the first button 104 and the second button 106 may be backlit. In other examples, the first actuator and/or the second actuator may be in the form of a switch which can be actuated by flipping the switch or any other suitable form of actuator.

The first button 104 is in the shape of a circle and comprises a smooth surface. The second button 106 is in the shape of a rectangle and comprises a bumpy surface. In this example, the first button 104 and the second button 106 therefore have different three-dimensional shapes, by their two-dimensional shapes and their different surface textures. The three-dimensional shape of the first actuator 104 or the second actuator 106 may include its externally visible two-dimensional shape as viewed by a user, such as a triangular, circular or rectangular button, and/or its surface texture including, for example, recesses and protrusions on the surface of the respective actuators. For example, the first actuator may have surface protrusions which spell “Pee” in Braille, and the second actuator may have surface protrusions which spell “Poo” in Braille. In other examples, the Braille may be on the case adjacent to the first button 104 and the second button 106. This would mean that the Braille would be readable even if one of the surfaces of the buttons was textured. The different shape of the first actuator and the second actuator may refer to the size of the actuator. For example, the first actuator 104 and the second actuator 106 may both be rectangular, but the first actuator 104 may have a smaller rectangle than the second actuator 106.

In other examples, the first button 104 and the second button 106 may have the same shape as each other, or may have any suitable three-dimensional shapes to denote their purpose.

The first button 104 may also have a different color to the second button 106, for example, the first button 104 may be yellow and the second button 106 may be brown. The shades of the colors may be selected by taking into account color blindness around the world by, for example, selecting shades of each color that least affects those with color blindness.

The different color and three-dimensional shape of the first button and the second button will make them easily distinguishable to the user, so that they know which of the first actuator 104 and second actuator 106 they need to actuate for their use.

As shown in FIG. 2, the device 100 comprises a memory 108 in which a plurality of audio files are stored. In this example, there are four audio files stored in the memory: a male urination file, a female urination file, a male defecation file and a female defecation file. The female urination file comprises a sound resembling a female urinating in a female toilet. The male urination file comprises a sound resembling a male urinating in a male toilet. The female defecation file comprises a sound resembling a female defecating in a female toilet. The male defecation file comprises a sound resembling a male defecating in a male toilet. This accounts for the variation in normal noises that a person might expect to hear in their usual encounters in public toilets.

The sound resembling a female urinating in a female toilet may include the sound which may be heard in the vicinity, such as within the stall area of a public toilet. The male toilet may include a urinal or a toilet in a stall. The sound resembling a male urinating in a male toilet may include the sound which may be heard in the vicinity, such as within a bathroom with urinals. The sound resembling a female defecating in a female toilet may include the sound which may be heard in the vicinity, such as within the stall area of a public toilet. The sound resembling a male defecating in a male toilet may include the sound which may be heard in the vicinity, such as within the stall area of a public toilet.

Each of the urination files and defecation files may be created from a recording of a person urinating or defecating, with any ambient sounds scrubbed out by a sound engineer. Each of the urination files (including the female urination file and the male urination file) and the defecation files (including the female defecation file and the male defecation file) may comprise an audio loop of the sound resembling a person urinating and defecating respectively. The audio loop may be created using Ambient Sound Engineering and/or Looping Audio Technology. This means that when the sound is played by a speaker, it can be played endlessly and seamlessly which may help to soothe the user, and thus aid them in relieving themselves.

In some examples, the memory 108 may comprise more or fewer than these four audio files. For example, when there is only a single actuator, there may be only a single audio file, such as a urination file comprising audio resembling a person urinating, or there may be only a single audio file such as a defecation file comprising audio resembling a person defecating. There may be both a single urination file and a single defecation file. In some examples, there may be a female file and a male file, such as a female urination file and a male urination file or a female defecation file and a male defecation file. In yet further examples, there may be multiple different options for female and male urination files and female and male defecation file, so that the user can choose their preferred sounds.

The device 100 comprises a controller 120 which controls the device speaker 102 to play one of the audio files when the first actuator 104 and the second actuator 106 are actuated. In this example, the default option on the device 100 may be that, when the first actuator 104 is actuated, the controller 120 controls the device speaker 102 to play the female urination file, and when the second actuator 106 is actuated, the controller 120 controls the device speaker 102 to play the female defecation file. In examples where there is only one urination file and one defecation file stored in the memory 108, the controller 120 may control the device speaker to play the urination file when the first actuator 104 is actuated, and may control the device speaker 102 to play the defecation file when the second actuator 106 is actuated.

The controller 120 in this example may control the device speaker 102 to play an audio file when one of the first and second actuator 104, 106 is depressed (i.e., actuated), and may control the device speaker 102 to stop playing the audio file when the respective actuator 104, 106 is depressed a second time. In this manner, the first actuator 104 and the second actuator 106 provides inputs to the controller 120. In examples where the first actuator 104 and/or the second actuator 106 is a switch, the controller 120 may control the device speaker 102 to play an audio file when a switch is flipped (i.e., actuated), and may control the device speaker 102 to stop playing the audio file when the respective switch is flipped back (i.e., actuated a second time). In other examples the controller 120 may control the device speaker 102 to play an audio file when one of the first and second actuator 104, 106 is depressed, and may control the device speaker 102 to stop playing the audio file when the respective actuator 104, 106 is released.

Where there are two or more actuators on the device 100, such as in this example, when more than one of the actuators 104, 106 are actuated simultaneously, the controller 120 may control the device speaker 102 as though only one of the actuators 104, 106 was actuated. On other words, the controller 120 may control the device speaker 102 to play only one of the urination file and defecation file. The controller 120 may determine whether the first actuator 104 or the second actuator 106 was actuated first, and play the corresponding audio file of the respective actuator which was actuated first. Once the actuator which was actuated first is released, the controller 120 may control the device speaker 102 to play the audio file corresponding to the actuator which was actuated second if the respective actuator is still actuated.

The device 100 in this example comprises a male selection actuator 110 and a female selection actuator 112. The selection actuators 110, 112 provide inputs to the controller 120. Actuating the male selection actuator 110 selects the male files in the memory 108 to be played, including the male urination file and the male defecation file. Actuating the male selection actuator 110 therefore results in the controller 120 controlling the device speaker 102 to play the male urination file when the first actuator 104 is actuated and to play the male defecation file when the second actuator 106 is actuated. Actuating the female selection actuator 112 selects the female files in the memory 108 to be played, including the female urination file and the female defecation file. Actuating the female selection actuator 112 therefore results in the controller 120 controlling the device speaker 102 to play the female urination file when the first actuator 104 is actuated and to play the female defecation file when the second actuator 106 is actuated. This can allow for easy switching between male files and female files.

In this example, the device 100 comprises a light 114 which the controller 120 controls to illuminate when the first actuator 104 has been actuated and/or when the second actuator 106 has been actuated. This gives a visual cue that the user should be hearing an audio file play on the device speaker 102, and may help the user to troubleshoot if they cannot hear the audio file playing.

In this example, the device 100 comprises a first volume adjusting actuator 116 and a second volume adjusting actuator 118. The controller 120 may control the volume of the device speaker 102 based on the actuation of the volume adjusting actuators such that the volume adjusting actuators 116, 118 provide inputs to the controller 120. The first volume adjusting actuator 116 and the second volume adjusting actuator 118 may be in the form of buttons, which may be in the shape of rectangles. The first volume adjusting actuator 116 may comprise a protrusion or a recess in the form of a line (or a minus sign), and the second volume adjusting actuator 118 may comprise a protrusion or recess in the form of a cross (or a plus sign). Actuating the first volume adjusting actuator 116 may reduce the volume of the device speaker 102, and actuating the second volume adjusting actuator 118 may increase the volume of the device speaker 102.

In this example, the device 100 may comprise a wireless technology device 122, such as a short-range wireless technology device 122, including a Bluetooth® device, a Near Field Communication (NFC) device, Wifi®, cellular technology, or any application programming interface (API). In this example, the wireless technology device 122 may be a Bluetooth® device. In other examples, the wireless technology device 122 may include WiFi®, cellular technology, or Near Field Communication (NFC) technology. A user may prefer for the audio files to be sent to a hearing aid, or to headphones or any other type of remote speaker. When headphones are connected to the Bluetooth® device 122, the controller 120 may, upon actuation of the first actuator 104 or the second actuator 106, control the Bluetooth® device 122 to transmit the respective audio file to the connected remote speaker, so that the remote speaker plays the respective audio file. In some examples, the device 100 may comprise multiple wireless technology devices, such as a Bluetooth® device, a WiFi® device, and a cellular technology device so that a remote speaker may be connected to the device 100 by multiple different technologies.

In some examples, the device 100 may link up with hearing aid suppliers by using shared application programming interfaces (APIs) to allow the audio files to be played directly by the hearing aid, rather than transmitted through a Bluetooth® device 122.

In this example, the device 100 comprises a power management module 124 which is configured to receive power from a power supply. The power supply in this example is in the form of a plug 126 which connects to a power outlet, and which transfers the power from the power outlet to the power management module 124. The plug 126 may have a third earthing pin or may not have a third earthing pin. The plug 126 may comprise a cable as shown in FIG. 1, or be mounted on the back of a casing of the device 100. The power management module 124 then distributes power to the other components within the device 100.

In this example, the device 100 may comprise a screen 128. The screen 128 may be an LCD screen, and may be used to show any useful information to the user. For example, it may be used to show whether the male files or the female files have been selected. It may show whether it is currently playing any of the audio files. The screen 128 may show the volume level of the device 100. In some examples, the screen 128 may be a touchscreen. In such examples, the actuators may be on the touchscreen, such that all controls and instructions may be received from a user through the touchscreen.

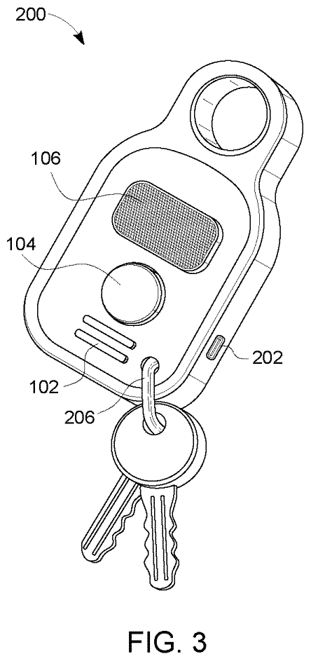

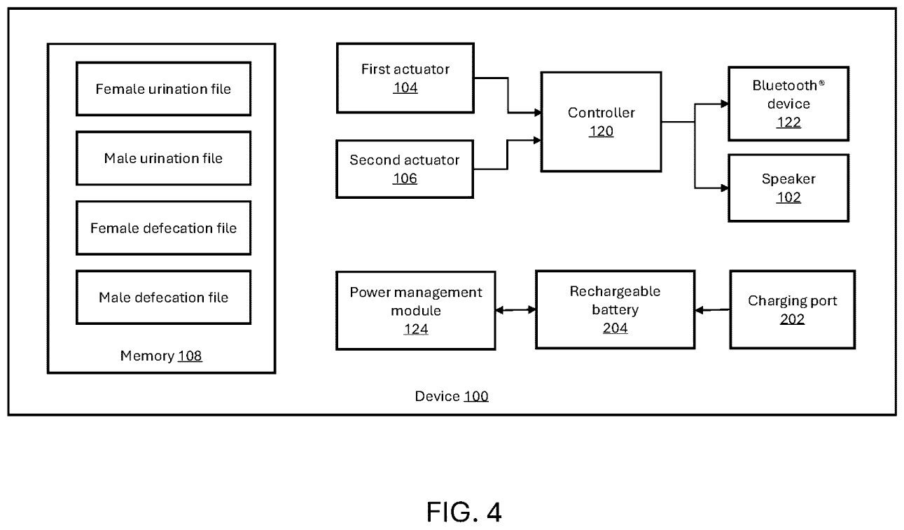

FIG. 3 shows a second example device 200 which may be similar to the first example device 100, with like parts being denoted by like reference numerals. FIG. 4 shows a block diagram of the components of the second example device 200. The second example device 200 may differ from the first example device 100 by being portable (in other words, mobile). It may comprise a case which is built from two halves that pair together to form a single case enclosure, containing all of the components of the device 200. The case may be ergonomically shaped to comfortably fit into a user's hand (i.e., their palm). The second example device 200 may therefore be useful in public restrooms, since it can be carried with the user.

The second example device 200 may be similar to the first example device in comprising a memory 108, a first actuator 104, a second actuator 106, a controller 120, a speaker 102, a power management module 124, and a Bluetooth® device 122.

The second example device 200 may differ from the first example device 100 in that it may require the first button 104 and the second button 106 to be depressed and held down for the controller 120 to control the device speaker 102 to play the respective audio file. This is to reduce accidental activations in pockets or purses in public.

The second example device 200 may differ from the first example device 100 in comprising a through-hole 204 for receiving a keyring, so that it can be attached to a set of keys. In this example, the second example device 200 may also comprise a second through hole 206 for receiving a second, separate keyring. The two through-holes may be on opposing sides of the device 200. Having two through-holes 204, 206 for receiving keyrings allows for one of the keyrings to be used for general keys, and the other keyring to be used to attach a bathroom key. For people suffering from conditions which require urgent access to bathrooms, having a separate keyring for a bathroom key, which is easily accessible, can be important to reaching a bathroom in time.

The second example device 200 may differ further from the first example device 100 in comprising a battery 204. In this example, the battery may be a rechargeable battery, such as a rechargeable Lithium-ion battery which may be in a variety of forms, such as in a coin shaped form or a soft-covered foil packet “pouch” battery. The second example device 200 may also comprise a charging port 202 for charging the rechargeable battery 204. In other examples, there may be a battery access hatch or door, through which batteries can be accessed and replaced. The power management module 124 in this example receives power from the battery 204, and distributes power to the remaining components within the device 200 as needed. In some examples, the battery 204 may be recharged through wireless charging. In such examples, there may still be a charging port 202 to enable charging through the port 202 or through wireless charging. In other examples, there may be no charging port 202.

The second example device 200 may differ from the first example device 100 in not comprising any selection actuators, or any volume adjusting actuators. Instead, the second example device 200 may comprise only the first actuator 104 and the second actuator 106 as inputs to the controller 120. Selection of the audio files and volume adjustment may still be carried out on the device 200 itself, but with the first actuator 104 and the second actuator 106. For example, the female files may be the default files to be played on the device when either the first actuator 104 or the second actuator 106 is actuated, but the male files may be selected by double clicking the first actuator 104 or the second actuator 106, or by depressing the first actuator 104 and the second actuator 106 in a predetermined sequence, rhythm or length of time. Where the controller 120 requires a second actuation of a respective actuator to control the device speaker 102 to stop playing the audio file or to control the Bluetooth® device 122 to stop transmitting the audio file to a remote speaker, holding down the first actuator 104 or the second actuator 106 for a predetermined length of time may toggle between the male files and the female files.

In other words, upon receiving a toggle signal from the first actuator 104 and/or the second actuator 106 which is different from the actuation of the respective actuator 104, 106, the controller 120 may switch between the female files and the male files, such that upon actuation of the first actuator 104 and the second actuator 106, either the respective female file or the respective male file is played on the device speaker 102. The controller 120 may control the device speaker 102 to play a confirmation sound to confirm that the toggle signal has been received and that the files have been switched. The confirmation sound may be for example, a beep, a double beep, a chirp, or any suitable sound. The confirmation sound may be different for switching from male files to female files than for switching from female files to male files. For example, the confirmation sound for switching from male files to female files may be a chirp whereas the confirmation sound for switching from female files to male files may be a double beep. This could give an audible cue to the user that the device 100 is primed to play the right audio files for that user. In some examples, the controller 120 may control the light 114 to illuminate in a flashing-light sequence, to act as a visual confirmation of the audible confirmations also activated at that time. This is to help those with hearing aids, whether or not their hearing aids are then connected via the wireless technology device 122, so that they can rely on seeing the light 114 flashing for their own confirmation that the switch between files has been performed. The color of the light 114 may be of one color, such as amber or red, or it may be an LED light that is able to display a range of colors, such as green, blue, yellow, etc. in addition to red and amber. If an LED that is capable of displaying numerous colors is used in the device 100, then the controller 120 may control it to display one color for one set of confirmations, such as green, for changing the gender from female to male or from male to female, while displaying a different color to confirm action on the other set of values to be programmed, such as red for changing the respective sound profiles for peeing or defecating within a gender.

There may be a default volume at which the controller 120 controls the device speaker 102 to play the audio files, or at which the controller 120 controls the Bluetooth® device 122 to transmit the audio files to the remote speaker. The volume may be adjusted by double clicking the first actuator 104 or the second actuator 106, to turn actuation of the first actuator 104 and the second actuator 106 into volume adjusting inputs to the controller 120, in a similar manner to the first volume adjusting actuator 116 and the second volume adjusting actuator 118 in the first example device 100. The inputs to the controller 120 of the first actuator 104 and the second actuator 106 may then be reverted back to their original states by double clicking the first actuator 104 or the second actuator 106 again. The first actuator and the second actuator may be converted into volume adjusting actuators with any suitable actuation pattern or sequence. When the file selection and volume adjustment are possible on the same device, then the actuation sequence may be different for toggling between male files and female files, and for using the first actuator 104 and second actuator 106 as volume adjusting actuators.

In other words, upon receiving a volume signal from the first actuator 104 and/or the second actuator 106 which is different from the actuation of the respective actuator, and different from the toggle signal, the controller 120 may consider further actuation of the first actuator 104 and second actuator 106 as volume adjusting actuations. Upon receiving the volume signal again from the first actuator 104 and/or second actuator 106, the controller 120 may consider actuations of the first actuator 104 and second actuator 106 to be their original function as playback actuators.

In some examples, it may be preferable to not be able to adjust the volume on the device at all. This may eliminate the possibility of inadvertently increasing the volume in a public restroom which may increase the anxiety associated with the users' conditions. Instead, volume adjustment may be carried out on an online portal, which may be accessed by connecting the device 200 to a computing device through the charging port 202, such as a USB-C charging port, or by connecting the device 200 to the computing device with the wireless technology device 122, such as the Bluetooth® device. On the online portal, it may be possible to adjust the volume of the device speaker 102 to the preferred volume. The controller 120 thereby receives a signal from the portal to adjust the volume of the device speaker 102 accordingly when an actuator 104, 106 is actuated.

On the online portal, it may also be possible to activate, set-up or authenticate the device 100. This may help to reduce counterfeit products since they would not be able to connect to the portal to authenticate or conduct any of the actions, such as adjusting device volume, from the online portal.

In some examples, it may also be preferable not to be able to select the files on the device. For example, the device 200 may be preloaded with only male files or only female files. In other examples, where file selection is necessary, this may be carried out on the online portal, where files may be selected and/or downloaded to the memory 108 of the device 200. The memory 108 in the device 200 may be preloaded with female files and male files and the user may be able to select, on the online portal, either the female files or the male files to be played when an actuator 104, 106 is actuated. The controller 120 therefore receives a signal from the online portal to select the female file or the male file, in accordance with the user's selection, to play on the device speaker 102 or to transmit through the Bluetooth® device 122 when an actuator is actuated. In other examples, there may be no audio files preloaded on the memory 108, and the files may be downloaded to the memory 108 through the online portal. In further examples, there may be audio files already stored in the memory 108, but there may be an option to select alternative files through the online portal, which can be downloaded to the memory 108, if they are preferred by the user.

The devices 100, 200 may also be used to help users to condition or train themselves to be able to respond to these audio cues to relieve themselves by classical conditioning or Pavlovian conditioning. Over time, users may therefore become more comfortable relieving themselves when hearing the respective audio cues.

The device 100, 200 includes hardware elements that can be electrically coupled via a bus (or may otherwise be in communication, as appropriate). The hardware elements can include one or more processors, including without limitation one or more general-purpose processors and/or one or more special-purpose processors (such as digital signal processing chips, graphics acceleration chips, and/or the like); one or more input devices; and one or more output devices, which can include without limitation a display device or the like.

The device 100, 200 may further include (and/or be in communication with) one or more storage devices, which can include, without limitation, local and/or network accessible storage and/or can include, without limitation, a disk drive, a drive array, an optical storage device, a solid-state storage device, such as a random access memory (“RAM”) and/or a read-only memory (“ROM”), which can be programmable, flash-updateable and/or the like. The device 100, 200 might also include a communications subsystem, which can include without limitation a modem, a network card (wireless or wired), an infrared communication device, a wireless communication device and/or chipset (such as a Bluetooth device, an 802.6 device, a Wi-Fi device, a WiMax device, cellular communication facilities, etc.), and/or the like. The communications subsystem may permit data to be exchanged with a network (such as the network described below, to name one example), and/or any other devices described in this document. In some embodiments, the device 100, 200 will further include a working memory, which can include a RAM or ROM device, as described above.

The device 100, 200 also can include software elements, including an operating system and/or other code, such as one or more application programs, which may include computer programs. A set of these instructions and/or codes might be stored on a computer-readable storage medium.

In some cases, the storage medium might be incorporated within the device 100, 200 or in communication with the device 100, 200. In other embodiments, the storage medium might be separate from a device 100, 200 (e.g., a removable medium, such as a compact disc, etc.), and/or provided in an installation package.

Although it has been described that the example device 100, 200 comprise no light, or one light 144, in some examples, the device 100, 200 may comprise more than one light 114. One or more of the lights may be illuminated to indicate that a hearing device, such as a hearing aid, earphones, or headphones is connected to the Bluetooth® device 122. One or more of the lights may be illuminated to indicate that a battery 204 has a low charge, and may need replacing or recharging soon. One or more of the lights may be illuminated to indicate that a battery 204 has sufficient charge to play or transmit the audio files, such that a lack of illumination would indicate to a user that the battery is not sufficiently charged to work. A single light may perform any combination of these functions, and may have different modes to indicate each of the functions, such as illuminating in different colors, being permanently illuminated, turning on and off in a predetermined pattern, flashing and/or changing the brightness in a predetermined pattern.

Although the second example device 200 has been described as not having some of the features described in the first example device 100, in other examples, the portable second example device 200 may have any combination of the features described with reference to the first example device 100.

It will be appreciated that this technology may be licensed to cellular original equipment manufacturers (OEMs), application (app) stores, or to key-fob device manufacturers such as automotive OEMs, for example.

Unless otherwise specified, the term “substantially” means within 5% or 10% of the value referred to or within manufacturing tolerances. Unless otherwise specified, the term “about” means within 5% or 10% of the value referred to or within manufacturing tolerances.

The conjunction “or” is inclusive.

The terms “first”, “second”, “third”, etc. are used to distinguish respective elements and are not used to denote a particular order of those elements unless otherwise specified or order is explicitly described or required.

Numerous specific details are set forth to provide a thorough understanding of the claimed subject matter. However, those skilled in the art will understand that the claimed subject matter may be practiced without these specific details. In other instances, methods, apparatuses or systems that would be known by one of ordinary skill have not been described in detail so as not to obscure claimed subject matter.

Some portions are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is a self-consistent sequence of operations or similar processing leading to a desired result. In this context, operations or processing involves physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained in software to be used in programming or configuring a computing device.

The use of “adapted to” or “configured to” is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included are for ease of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations and/or additions to the present subject matter as would be readily apparent to one of ordinary skill in the art.

That which is claimed:

1. A device for assisting a user to relieve themselves, the device comprising:
a memory in which at least one audio file is stored, the at least one audio file including a urination file comprising audio resembling a person urinating and/or a defecation file comprising audio resembling a person defecating;
a device speaker;
an actuator to be actuated by a user, and
a controller that, upon actuation of the actuator, controls the device speaker to play the urination file and/or the defecation file.

2. The device according to claim 1, wherein the urination file comprises an audio loop of a person urinating and/or the defecation file comprises an audio loop of a person defecating.

3. The device according to claim 1, wherein:
the actuator is a first actuator,
the at least one audio file comprises the urination file and the defecation file, and
the device comprises a second actuator to be actuated by the user, wherein the controller:
upon actuation of the first actuator, controls the device speaker to play the urination file; and
upon actuation of the second actuator, controls the device speaker to play the defecation file.

4. The device according to claim 3, wherein the first actuator comprises a different shape and/or color to the second actuator.

5. The device according to claim 1, wherein the at least one audio file is played on the device speaker while the actuator is actuated and stopped when the actuator is released.

6. The device according to claim 1, wherein the at least one audio file is played on the device speaker when the actuator is actuated and stopped with the actuator is actuated a second time.

7. The device according to claim 1, wherein the at least one audio file comprises:
a female file comprising a female urination file and/or a female defecation file, the female urination file comprising a sound resembling a female urinating in a female toilet and the female defecating file comprising the sound resembling a female defecating in a female toilet; and
a male file comprising a male urination file and/or a male defecation file, the male urination file comprising a sound resembling a male urinating in a male toilet and the male defecating file comprising a sound resembling a male defecating in a male toilet.

8. The device according to claim 7, wherein the controller, upon receiving a signal from the actuator which is different from the actuation of the actuator, switches between the female file and the male file, such that upon actuation of the actuator, either the female file or the male file is played on the device speaker.

9. The device according to claim 1, wherein the controller is connected to a portal with a wireless technology device disposed on the device, wherein the controller receives a signal from the portal to select alternative audio files to be stored on the memory.

10. The device according to claim 1, wherein the controller is connected to a portal with a wireless technology device disposed on the device, wherein the controller receives a signal from the portal to select the volume of the device speaker.

11. The device according to claim 7, wherein the controller is connected to a portal with a wireless technology device disposed on the device, and wherein the controller receives a signal from the portal to select the female file or the male file to be stored on the memory.

12. A device for assisting a user to relieve themselves, the device comprising:
a memory in which at least one audio file is stored, the at least one audio file including a urination file comprising audio resembling a person urinating and/or a defecation file comprising audio resembling a person defecating;
a wireless technology device;
an actuator to be actuated by a user, and
a controller that, upon actuation of the actuator, controls the wireless technology device to transmit the at least one audio file to a remote speaker.

13. The device according to claim 12, wherein the urination file comprises an audio loop of a person urinating and/or the defecation file comprises an audio loop of a person defecating.

14. The device according to claim 12, wherein:
the actuator is a first actuator,
the at least one audio file comprises the urination file and the defecation file, and
the device comprises a second actuator to be actuated by the user, wherein the controller:
upon actuation of the first actuator, controls the wireless technology device to transmit the urination file to the remote speaker; and
upon actuation of the second actuator, controls the wireless technology device to transmit the defecation file to the remote speaker.

15. The device according to claim 14, wherein the first actuator comprises a different shape and/or color to the second actuator.

16. The device according to claim 12, wherein the at least one audio file comprises:
a female file comprising a female urination file and/or a female defecation file, the female urination file comprising a sound resembling a female urinating in a female toilet and the female defecating file resembling the sound of a female defecating in a female toilet; and
a male file comprising a male urination file and/or a male defecation file, the male urination file comprising a sound resembling a male urinating in a male toilet and the male defecating file resembling the sound of a male defecating in a male toilet.

17. The device according to claim 16, wherein the controller, upon receiving a signal from the actuator which is different from the actuation of the actuator, switches between the female file and the male file, such that upon actuation of the actuator, either the female file or the male file is played on the device speaker.

18. The device according to claim 12, comprising two through holes, each through hole for receiving a respective keyring.

19. The device according to claim 12, wherein the device comprises a rechargeable battery and a charging port for charging the battery.

20. A device for assisting a user to relieve themselves, the device comprising:
a memory in which at least two audio files are stored, the at least two audio files including a urination file comprising audio resembling a person urinating and a defecation file comprising audio resembling a person defecating;
a wireless technology device;
a device speaker;
a first actuator to be actuated by a user and a second actuator to be actuated by a user, wherein the first actuator comprises a different three-dimensional shape and/or color to the second actuator, and
a controller that:
upon actuation of the first actuator, controls the device speaker to play the urination file or controls the wireless technology device to transmit the urination file to a remote speaker, and
upon actuation of the second actuator, controls the device speaker to play the defecation file or controls the wireless technology device to transmit the urination file to the remote speaker.
